(19) United States
(12) Patent Application Publication
(10) Pub. No.: US 2025/0166817 A1
(43) Pub. Date: May 22, 2025

(54) ADAPTIVE INTERACTION BETWEEN SMART HEALTHCARE SYSTEMS

(71) Applicant: Cilag GmbH International, Zug (CH)

(72) Inventors: Frederick E. Shelton, IV, Hillsboro, OH (US); Jason L. Harris, Lebanon, OH (US); Jacqueline Corrigan Aronhalt, Loveland, OH (US)

(21) Appl. No.: 18/810,036

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. G16H 40/67

(52) U.S. Cl. CPC G16H 40/67

(57) ABSTRACT

A surgical smart system for an operating room may collect data on surgical procedures and equipment. Upon identifying specific surgical equipment, the surgical smart system may present various interaction levels—minimal, intermittent, or full. The system may dynamically choose an interaction level based on a surgical manifest and equipment use. The system may monitor the equipment's performance, adjusting interaction levels based on equipment usage patterns and its impact on patient physiology. Operational data may be displayed on an interface and archived in a database, creating a digital record of the equipment's use during surgery.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

This application is related to the following, filed contemporaneously, the contents of each of which are incorporated by reference herein:
U.S. patent application Ser. No. 18/810,323, filed Aug. 20, 2024.

### BACKGROUND

With the complexity and autonomy of smart devices, particularly in the medical field, interactions may be managed between multiple smart devices (e.g., and legacy devices). Systems may operate in isolation or with limited collaboration, limiting their effectiveness and potentially leading to instability or predictability failures. Means for coordinating these systems may be static and may not adapt based on changing circumstances or patient parameters, posing a potential challenge in providing patient care and monitoring.

### SUMMARY

Within healthcare, systems may facilitate an environment conducive to medical practices. A device may enable interaction, coordination, and control among one or more smart and/or legacy system. By implementing algorithms (e.g., dynamic algorithms) and methodologies, the device may adapt system behavior based on variables, conditions, and/or parameters. The operation of the device and interaction between the device, smart system, and/or legacy system may affect (e.g., enhance) the collective performance of the device, smart system, and/or legacy smart system.

The device may include a decision-making mechanism that ascertains whether and how two or more systems may interact (e.g., under varying circumstances). The decisions consider variables, and the variables may include the systems' capacities to cooperate, considerations for data exchange, interrelationships of variables, and prioritization of patient or surgeon parameters (e.g., patient or surgeon needs). When the device recognizes the interdependence of closed-loop variables, the device may transition from a state of cooperation to a state of bidirectional open-loop communication (e.g., in order to safeguard system stability and patient safety).

The device may prevent system instability and predictability failures (e.g., non-correlated predictability failures). Real-time data related to patient conditions and system parameters may be used, and the real-time data of the interaction level between the systems may be adjusted. The device may identify and adapt to an instability cascade failure involving a patient monitoring smart system, a ventilation/sedation system, and/or a heating system. The device may manage non-correlated predictability failures by switching from global control to local control based on a comparison of an energy input rate and a heat bloom expansion rate.

The device may engage with legacy systems. The device may identify features compatible with the legacy system (e.g., employing various sensors and cameras), such as a USB port and/or a wireless connection, and guide a user (e.g., surgeon, operating room (OR) staff to connect the two). The device may control the legacy system or display data from the legacy system on an interface, affecting the user's ability to monitor and/or control a situation arising in the operating room or within a hospital environment.

The device may be integrated with interconnected medical technologies and/or platforms. The user controls of one system may be displayed on the device, imaging and control interfaces between systems may be transferred to the device, synchronized motion of multiple devices may be executed, cooperative interactions among devices may be executed and/or initiated.

### BRIEF DESCRIPTION OF DRAWINGS

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 shows an example surgical instrument.

FIG. 6 illustrates a system diagram of the operation of an Operating Room (OR) smart system, with, for example, a surgeon, data center, bed, patient, legacy device, and surgical systems data set.

FIG. 7 illustrates an example architecture diagram of a smart system.

FIG. 8 illustrates an example system diagram of an operating room smart system connected to legacy devices, alongside a surgical systems database.

FIG. 9 illustrates an example database decision-making process within pre-operative, intra-operative, and post-operative modules.

FIG. 10 illustrates an example block diagram of a surgical systems data set.

FIG. 11 illustrates an example of a smart system interfacing with an identified device.

FIG. 12 illustrates and example of a surgical smart system gathering and displaying information from an identified device.

FIG. 13 illustrates an example block diagram of a surgical smart system.

FIG. 14 illustrates an example block diagram of a surgical smart system.

### DETAILED DESCRIPTION

A more detailed understanding may be had from the following description, given by way of example in conjunction with the accompanying drawings.

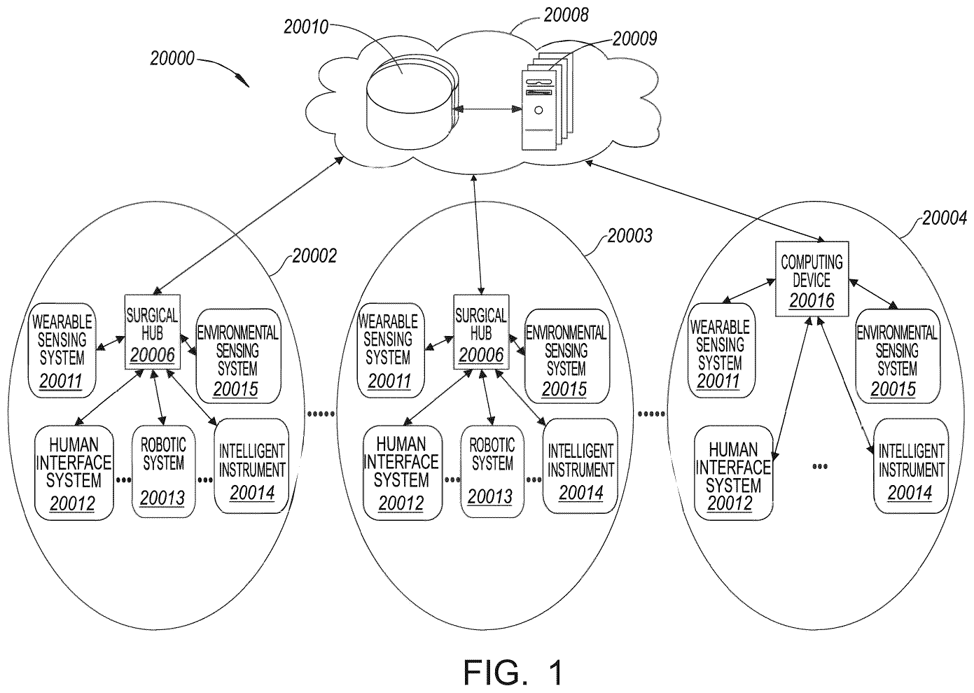

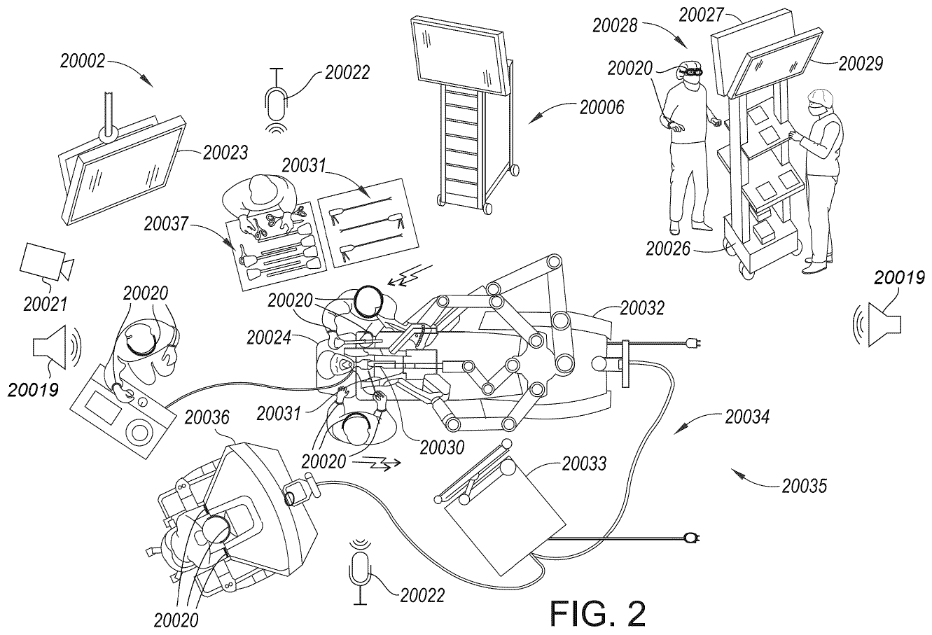

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

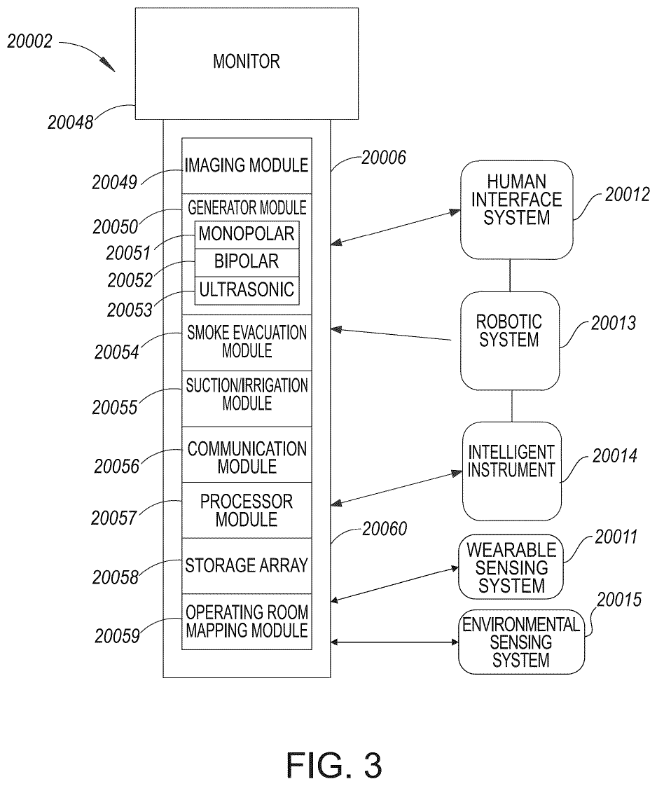

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

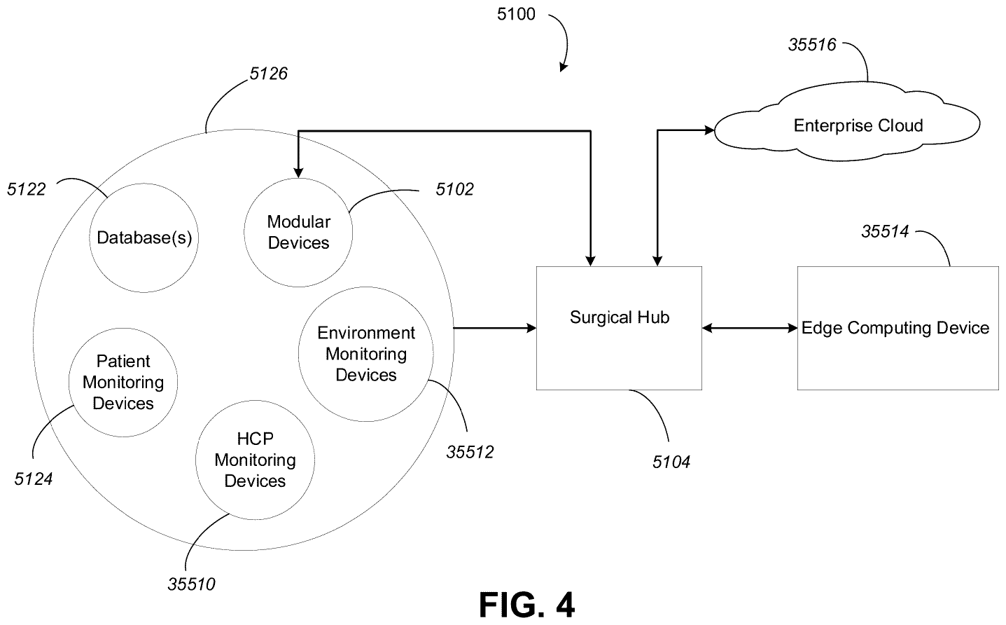

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient moni-toring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

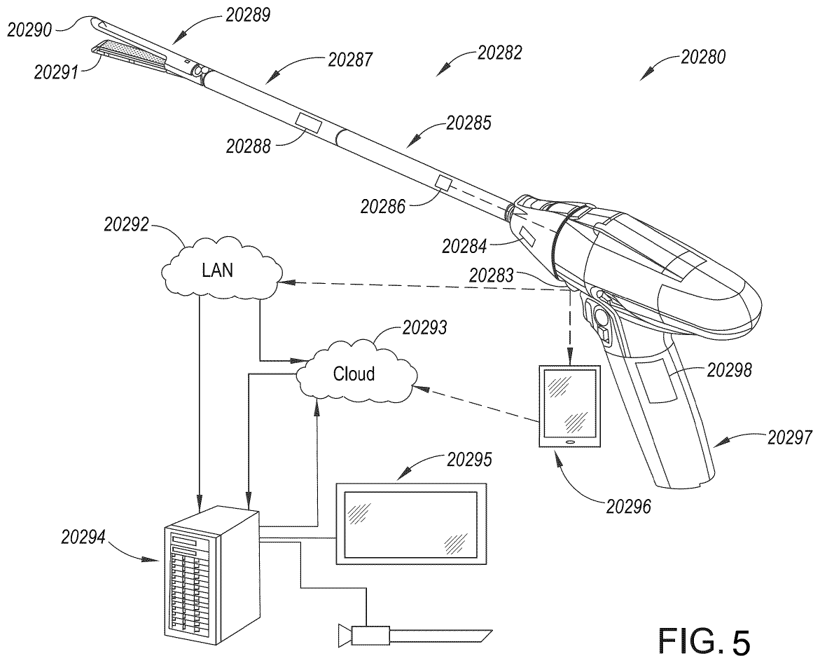

FIG. 5 illustrates an example surgical system 20280 that may include a surgical instrument 20282. The surgical instrument 20282 can be in communication with a console 20294 and/or a portable device 20296 through a local area network 20292 and/or a cloud network 20293 via a wired and/or wireless connection. The console 20294 and the portable device 20296 may be any suitable computing device. Surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

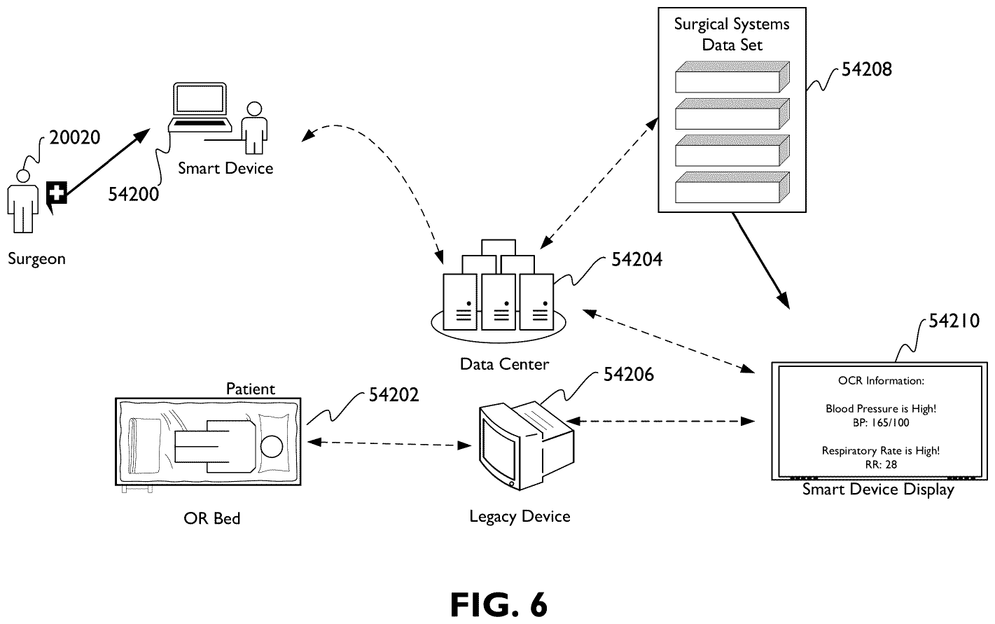

FIG. 6 illustrates an integrated surgical environment and depicts interconnectivity of legacy and smart systems within a medical procedure.

The surgeon 20020 may interface with various items during the surgical procedure. The surgeon 20020 may be referred to herein as the medical practitioner. The surgeon 20020 may interface directly with the smart device 54200 and may suggest an interactive system where the surgeon may input surgical parameters or procedural protocols. The smart device 54200 may provide real-time feedback or surgical guidance based on the data it (e.g., the smart device) processes.

The smart device 54200 may have a bidirectional communication channel with data center 54204. A (e.g., synchronous) data exchange may exist in the smart device 54200, wherein the smart device 54200 may transmit procedural metrics or receive updates based on system algorithms or surgical guidelines stored within the data center 54204.

Regarding the smart device's adaptability, the smart device 54200 may interface with a legacy device 54206 through a bidirectional link. The connection between the smart device 54200 and the legacy device 54206 may indicate that the smart device 54200 may integrate with and/or adapt to surgical equipment within the operating room. The smart device 54200 and the legacy device 54206 may be compatibility and expand utility across a broad range of medical settings.

The data center 54204 may not be a passive data repository. With a bidirectional link to the surgical systems data set 54208, the data center 54204 may query the dataset for relevant surgical protocols and updating the surgical systems data set 54208 with data (e.g., to refine procedural algorithms or record surgical outcomes).

The smart device display 54210 may be (e.g., directly) connected to the data center 54204 through a bidirectional channel and act as a visual interface for the smart device 54200. The smart device display 54210 may (e.g., dynamically) present data-driven insights, procedural guidance, or real-time metrics derived from the data center 54204. Direct connectivity to the Legacy device 54206 may enable the display to visually represent metrics or statuses from surgical systems. Legacy equipment (e.g., non-smart equipment) may be integrated into a feedback loop as described herein.

The surgeon's bidirectional communication with the OR bed may indicate a dynamic interface where the surgeon may adjust patient positioning with the potential to receive direct feedback on patient vitals or procedural progress, thereby modifying (or adapting to) the surgical process.

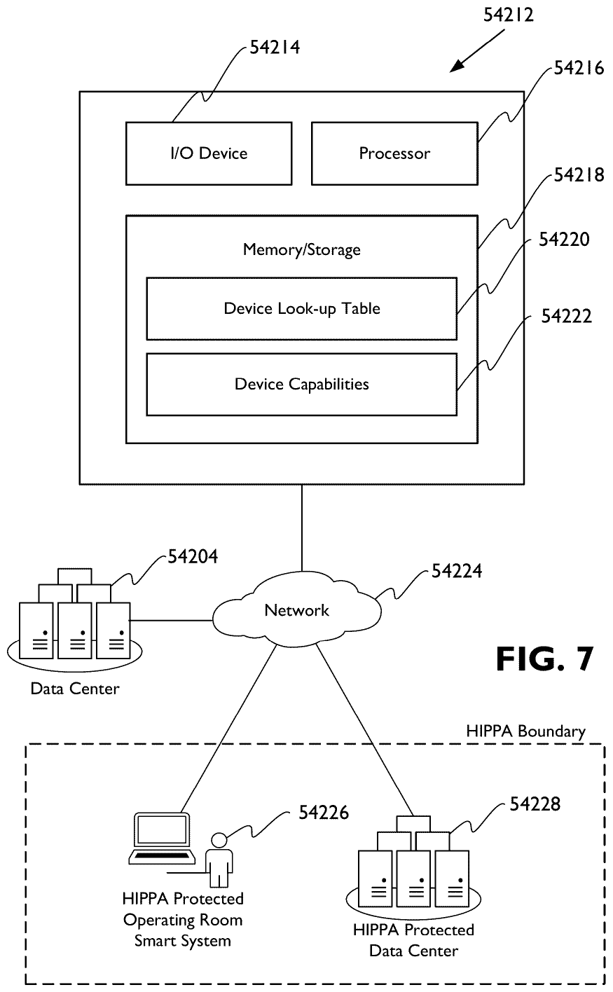

FIG. 7 illustrates an architecture diagram outlining the configuration of a surgical smart system operating within a HIPAA-protected operating room. The architecture may facilitate surgical procedures while upholding patient data privacy regulations.

The Input/Output (I/O) device 54214 may function as an intermediary that establishes communication between the surgical team and the smart system. Equipped with controls, dynamic displays, and interactive interfaces, the I/O device 54214 may enable real-time communication, allowing surgical personnel to issue commands, receive feedback, and access data during surgical procedures.

Embedded within the smart system device, a processor 54216 may operate as a computational hub, enabling task management, data processing, and system coordination. The processor 54216 may adeptly manage incoming data streams, execute algorithms, and synchronize component interactions, such that task execution and responsiveness may not be compromised.

The memory and storage 54218 within the device may serve as repositories for essential data, software applications, and surgical records. The memory and storage 54218 may house a device look-up table 54220, which may include information about surgical equipment and interaction configurations that are compatible with existing surgical equipment. The memory and storage 54218 may retain historical data, usage patterns, and operational logs for informed decision-making, predictive analytics, and continuous process modification.

Within the memory/storage device 54218, the device look-up table 54220 may compile an exhaustive repository of available surgical equipment, encompassing information such as specifications, compatibility criteria, usage guidelines, and interaction preferences of devices (e.g., legacy devices). The repository may be used for equipment selection and interaction customization, enabling precision and modification of (and for) surgical procedures.

The device capabilities 54222 (e.g., complementary to the device look-up table) may offer insights into the functionalities of surgical instruments. The device capabilities 54222 may be used for tailoring interaction levels in alignment with surgical requirements and modifying procedural efficiency by enabling equipment to be employed in the most suitable manner (e.g., as suggested by the smart surgical device 54200.

The data center 54204 may serve as a hub for data processing, storage, and analytics. The data center may host an expansive database including aggregated operational data, patient records, and procedural insights. Analytics, machine learning algorithms, and predictive modeling may be used to generate actionable insights for process refinement.

The network 54224 may be interconnected with the architecture components to facilitate data exchange and communication. The network may enable the transmission of operational data, alerts, and performance updates across the smart system device, the data center, and interconnected systems, supporting remote monitoring, collaboration, and informed decision-making during the surgical procedure.

In order to address data security and patient privacy concerns, the architecture may incorporate a HIPAA-protected operating room smart system 54226, adhering to regulatory healthcare standards. This system 54226 may enable the confidentiality of patient data while enabling surgical procedures.

Mirroring the operating room system's security, the data center may be HIPAA-protected 54228, assuring confidentiality and compliance of patient data stored within repositories of the data center. The dual layer of protection may uphold secure management of patient records and operational data throughout use of the data center 54228.

In smart systems, including in medical applications, the capability for autonomous operation may be described herein. The systems may achieve performance through strategic interaction. The interaction may be governed by a multifaceted decision-making process, which may be contingent upon questions: Can the systems interact? Should the systems interact? Is it beneficial for both or just one of the systems to engage in the interaction? Would the integration compromise the safe operation of the systems?

The circumstances that define how two smart systems interact are based on factors intrinsic to the systems themselves—can they or should they cooperate at a specific level? The factors may be delineated by the information a system uses to operate in a closed-loop format and whether both systems include closed-loop operation for optimal functionality. The interrelation between the systems may be governed by constraints based on the nature of the data exchanged, the interrelationship of the variables involved, or the relative importance of one system over the other as determined by an operator, such as a surgeon.

Examples may include an algorithm designed to manage an instability cascade failure. Examples may include a patient monitoring smart system, which may keep track of oxygen levels, carbon dioxide levels, and temperature. The vital(s) parameters may be shared with both the patient heating system and the ventilation/sedation systems. The ventilation/sedation system may use the patient's temperature and gas levels to adjust the sedation medication rate, tidal volume, and oxygen supplementation. The heating system may utilize the temperature data to modulate thermal load input. The interplay of the systems may include the patient's metabolism—affected by factors like sedation uptake and oxygen consumption—which may be temperature-dependent. A loop of interaction may be included, where the patient heating system, responding to temperature data, may indirectly influence the patient's metabolism. The modification in metabolism may impact the ventilator's function, which may attempt to maintain a stable sedation level. The modification may lead to an oscillating and unstable dynamic in both systems, which may be associated with a shift from cooperative interaction to a bi-directional open-loop communication mode.

Examples of system interaction may include a non-correlated predictability failure. A flexible endoscopic robot (e.g., a Type D smart device), may be used for the (e.g., precise) placement and control of RF needles during lung tumor ablation. The process may be monitored by an (e.g., advanced) visualization system, which may track the local external lung parenchyma temperature. Examples may include inhomogeneity in the density and conductivity of both the tumor and the parenchyma. The tumor's inhomogeneity may stem from its internal growth patterns, and the parenchyma's variation may be attributed to factors such as adhesion and chronic tissue remodeling. The disparity may lead to a misinterpretation by the laparoscopic camera, which may underestimate the heat penetration during ablation due to dense adhesions, to (e.g., to then) detect a sudden increase in temperature. The increase may not be due to a change in the ablative needles' activity and (e.g., and rather) a shift in the parenchyma's density. Examples may include a comparison between the energy input rate and the rate of heat bloom expansion. If the energy input rate and the rate of heat bloom expansion variables are found to be uncorrelated to a significant degree, the system may transition from relying on global temperature control data to a local bi-directional data exchange focused on the specific measurements.

The decision to maintain or alter the operational state of these systems may be based on circumstances surrounding the patient or a procedural occurrence. The determination may be triggered when a parameter related to either the patient or the operational device, based on a tissue parameter, deviates outside a predetermined acceptable range (e.g., a threshold). Parameters that may trigger such a shift in operational state may include heart rate, heart rate regularity, variability in heart rate, levels of oxygen or carbon dioxide in the blood, blood pressure, changes in correlations between two measurements of essentially the same patient variable (e.g., core temperature compared to extremity temperature, local temperature versus visualization temperature, etc.), tidal volume, pressures during inhalation or exhalation, blood sugar levels, and various inflammation indicators like Erythrocyte Sedimentation Rate (ESR), C-reactive protein (CRP), Plasma Viscosity (PV), and/or heart damage indicators including Cardiac Troponin, Creatinine Kinase (CK), CK-MB, and/or Myoglobin.

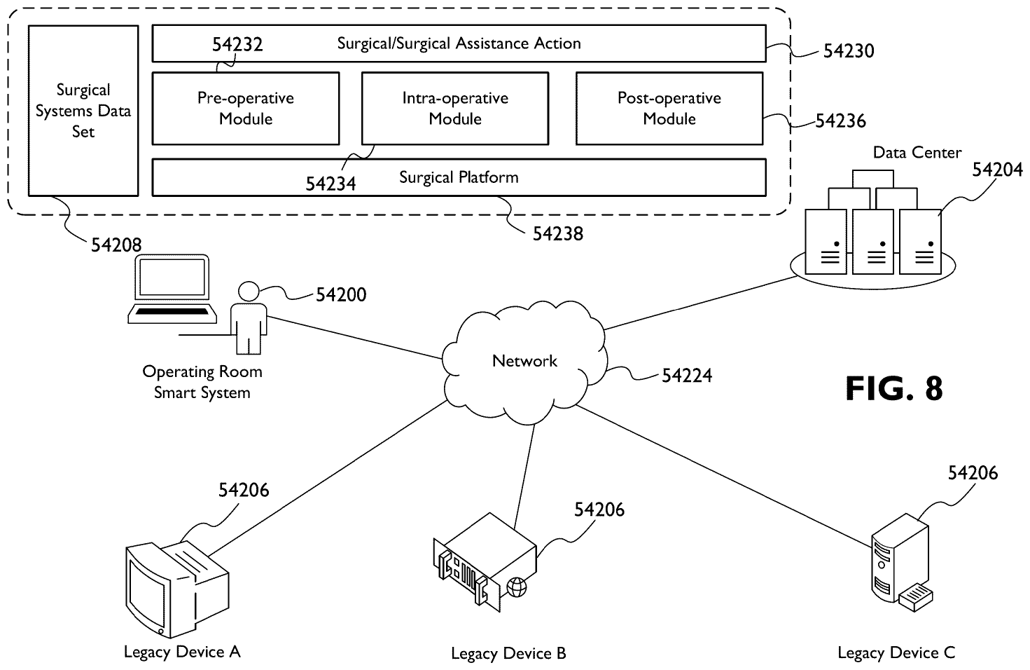

FIG. 8 illustrates a system diagram of an example configuration of an operating room smart system 54200 designed to interface, for example, with legacy devices. The capabilities of legacy surgical instruments may be bridged with smart systems.

The surgical systems data set 54208 may be a repository for data related to surgical protocols, equipment configurations, patient information, and more. Through the repository, a smart system may access data to inform its operations across multiple surgical phases.

In examples, alongside the surgical systems data set may be the surgical/surgical assistance action 54230. The surgical/surgical assistance action 54230 may guide or direct actions within the surgical environment. The surgical/surgical assistance action 54230 may operate by interfacing with the data set, extracting information, and providing instructions or support based on that data to either surgical instruments or medical personnel during procedures.

In examples, running parallel to the surgical/surgical assistance action may be the surgical platform 54238. The platform may oversee the integrated system, enabling coordination and operation of individual components and modules.

Three distinct modules are depicted nestled between the Surgical/Surgical Assistance Action 54230 and the Surgical Platform 54238 as follows.

The pre-operative module 54232 may facilitate preparatory activities leading up to the surgical procedure. Functions may include planning, device calibration, and patient preparation.

The intra-operative module 54234 may conduct actions facilitating the surgical procedure. The actions may include the guidance of surgical maneuvers, equipment monitoring, real-time decision-making support, etc.

The post-operative module 54236 may facilitate actions after the surgical procedure. The post-operative module 54236 may handle activities related to post-surgery care. The post-operative module 54236 may oversee patient monitoring, data storage, equipment cleanup, and analysis.

The Network 54224 may facilitate communication and data transfer among components. The network 54224 may connect the operating room smart system 54200 with the data center 54204, enabling an exchange of information. Furthermore, the connections to legacy devices, denoted as legacy device A (54206), B (54206), and C (54206), may allow the smart system to integrate the functionalities of the older devices into the current surgical workflow. The integration may confirm that no existing capability is unused or overlooked in the updated system (e.g., the system after recognizing legacy devices).

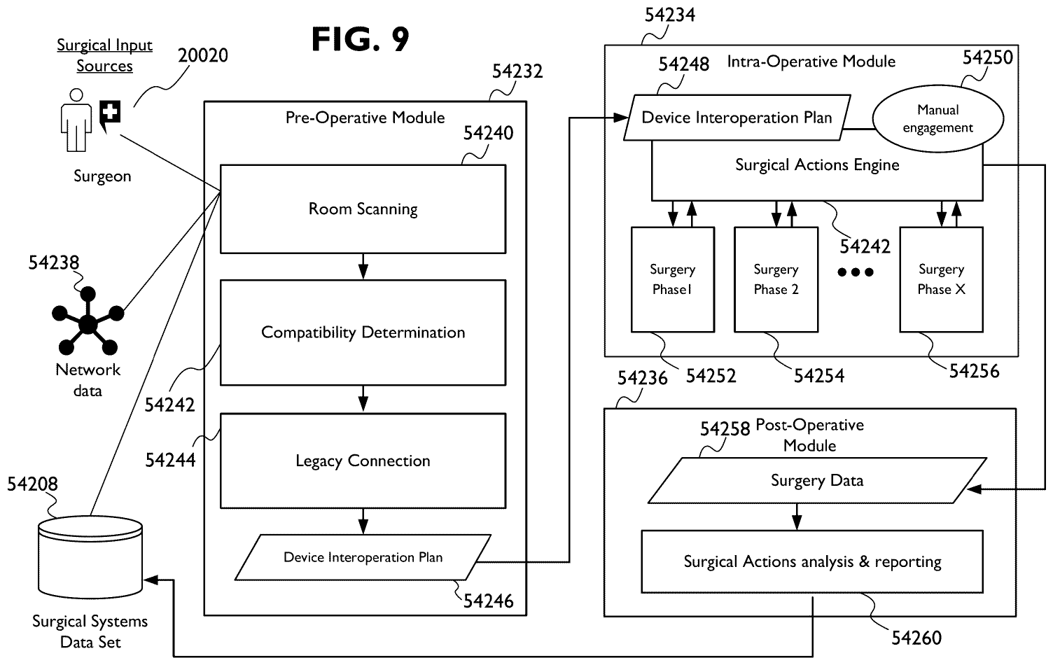

FIG. 9 illustrates an example database decision-making process within pre-operative, intra-operative, and post-operative modules.

The pre-operative module 54232 may have cooperative interactions with various integral components for preparing the surgical environment and enabling surgical planning. The pre-operative module 54232 may communicate with components like room scanning at 54240, aiding in capturing spatial details of the surgical environment, for example, in real-time. The interactions may involve extracting high-resolution images, determining spatial positions of surgical equipment, and identifying legacy systems present within the environment.

At 54242, a compatibility determination may be performed, for example, within the pre-operative module 54232. The compatibility determination may be programmed to interface with surgical devices having compatibility specifications, functional capabilities, and operating parameters. The compatibility determination may deduce effective communication strategies (e.g., for devices categorized as legacy), minimizing potential operational conflicts during surgical procedures.

At 54244, to enable legacy devices to be integrated with the smart system, a legacy connection may be initiated with communication protocols tailored for the devices (e.g., between the smart device and the legacy device). The legacy connection may (e.g., dynamically) determine the data transmission protocol, initiate handshake processes with devices, and communicate using software drivers designed for the specific legacy devices.

The pre-operative preparations may be orchestrated by the device interoperation plan at 54246, which may be tailored based on prior compatibility checks and environmental scanning. The interoperation plan while nested within the pre-operative module, may produce outputs, as indicated by 54248, may be relevant in surgical modules.

In the intra-operative module at 54234, the device interoperation plan, at 54248, may serve as a guide, for example, offering real-time command sequences. The relationship with the surgical actions engine 54242 may indicate a continuous feedback mechanism. The surgical actions engine 54242 may integrate decision-making algorithms, real-time data processing, and sensor-driven insights to interface between surgical phases ranging from surgery phase 1 to surgery phase x.

At 54250, manual engagement within the intra-operative module 54234 may allow surgical professionals to exercise discretion. Surgeons, through this component, may have the ability to modify or override the pre-determined operation plan, addressing real-time surgical nuances and unforeseen challenges that may arise during the surgical operation.

The system may consolidate surgical data at 54258. The data may capture details, from device interactions, surgical maneuvers, patient responses, to ambient conditions during the procedure, offering a data-driven perspective of the entire surgical event.

The post-operative module 54258 may use data analysis and insight extraction. Surgical actions analysis and reporting, at 54260, may employ computational models, comparative analytical techniques, and machine learning algorithms to interpret and process the data from the surgery data 54258. By interfacing with the surgical systems data set 54208, the post-operative module may derive actionable insights, retrospective evaluations, and predictive markers, adjusting/modifying surgical procedures in future surgical procedures.

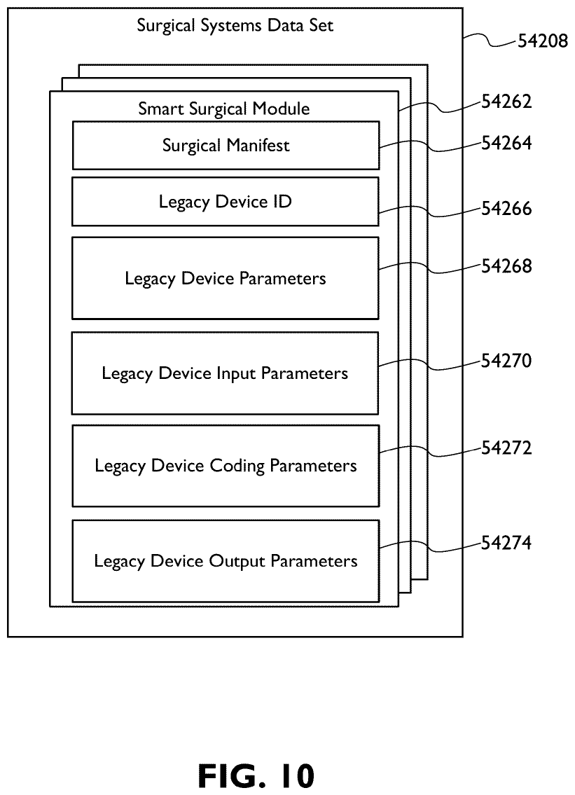

FIG. 10 illustrates an example of the surgical systems data set 54208. Within this data set, there may be the smart surgical module 54262. The smart surgical module 54262 may serve as an integrated component that houses and manages subsets of surgical data and related parameters.

The surgical manifest 54264 may function as a catalog or repository, for example, containing (e.g., detailed) records associated with surgical tools, procedures, and methodologies. The manifest may facilitate data retrieval and analysis for surgical professionals, allowing the surgical professionals to access relevant instrument-related information. The manifest may also be used to help detect legacy devices.

The legacy device ID 54266 may offer an identification mechanism for a legacy device, allowing for tracking and management of the legacy device. By equipping a legacy device with a unique identifier, interactions and data retrieval related to the device within a surgical or clinical environment may be streamlined.

The legacy device parameters 54268 may be a part of the smart surgical module 54262. The parameters may encompass a range of technical attributes associated with the legacy devices. The parameters may detail operational aspects such as device dimensions, functional thresholds, and other technical characteristics. By centralizing the parameters, the smart surgical module 54262 may provide an overview of a device's technical capabilities.

The legacy device input parameters 54270 may specify the nature and format of data inputs that a legacy device is configured to receive. Understanding the input parameters may enable data to be provided to the legacy device such that the data provided aligns with operational parameters of the legacy device.

The smart surgical module 54262 may include the legacy device coding parameters 54272. The parameters may relate to the coding or programming instructions tailored for a legacy device. By delineating the coding requirements and associated algorithms, the parameters may offer insight into the device's computational operations and its integration potential with other systems.

The smart surgical module 54262 may include the legacy device output parameters 54274. The legacy device output parameters may detail the nature, format, and structure of data outputs generated by the legacy device post-operation. Interpreting the output parameters may done by the interfacing system, enabling the output data or feedback from the device to be processed (e.g., accurately) in surgical stages.

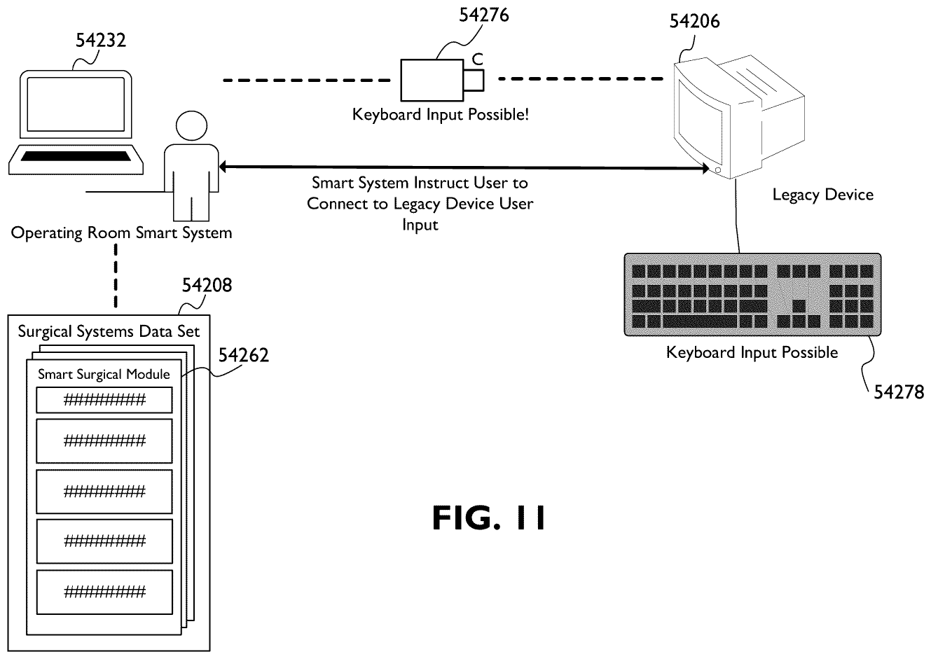

FIG. 11 illustrates an example configuration wherein the operating room smart system 54232 interfaces with interdependent components. FIG. 11 illustrates the system's capability to interact and interface with diverse devices, specifically legacy devices, and harness their functionalities.

The operating room smart system 54232, as depicted, may be in direct or indirect communication with the surgical systems data set 54208. Within this dataset may reside the smart surgical module 54262, potentially acting as an information repository. The smart surgical module 54262 may store an array of surgical metadata, encompassing specifics related to surgical instruments, protocols, and for example, legacy devices.

The operating room smart system 54232 may interface with a camera 54276. The camera 54276 may be embedded with optical and analytical capabilities and may monitor the surgical room's spatial area. The camera 54276 may detect, analyze, and categorize the features and functionalities of legacy devices 54206 within its view.

Through integration of (e.g., advanced) algorithms, the camera 54276 may discern the presence of legacy devices 54206 and their functionalities. In examples, upon inspection of a legacy device 54206, the camera 54276 may detect the possibility of keyboard input at 54278. The detection may be facilitated through pattern recognition algorithms or visual markers that are identifiable on the legacy devices by the camera 54276.

The operating room smart system 54232 may autonomously, or upon command, generate prompts or directives for the surgical staff. The directives may include actions that bridge the communication gap between the detected legacy device and the operating room smart system 54232 (e.g., instructing the staff to connect, via a cable/wireless communication medium, the operating room smart system 54232 to the legacy device 54206). The communication channel may enable data transfer, command execution, or remote manipulation of the legacy device.

Recognizing the keyboard input capabilities of the detected legacy devices 54206 may permit the operating room smart system 54232 to introduce or modify interactive mechanisms. In examples wherein manual input is possible, the system may use the legacy device's keyboard input, thus facilitating command sequences or data input operations.

The operating room smart system 54232 may interface with systems or sub-systems not illustrated in FIG. 11. In examples, the smart system may communicate with remote servers or data centers to harness computational power or storage capabilities, such as cloud computing. The interactions may be used when processing data streams in real-time or when synchronizing data across multiple surgical platforms.

The operating room smart system 54232 may support diverse communication protocols. Whether via wired connections or wireless transmission, including GSM, LTE, Bluetooth, WiFi, the system may maintain communication with peripheral devices or remote systems.

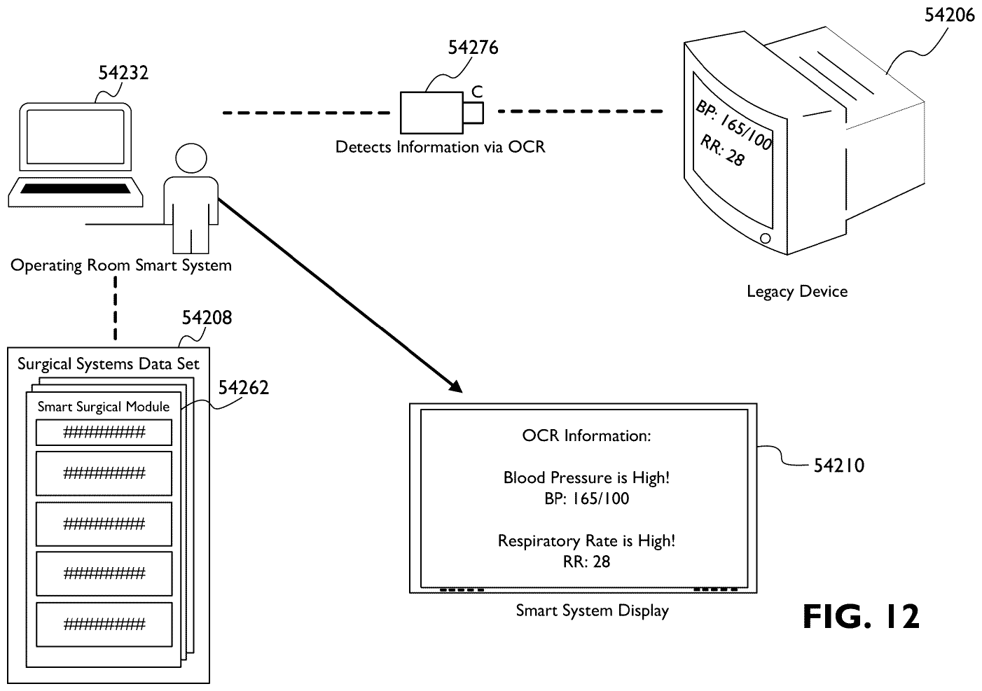

FIG. 12 illustrates a configuration emphasizing the data acquisition and display capabilities of the operating room smart system 54232 in relation to legacy devices. The architectural framework may demonstrate visualization and information synthesis, contributing to informed surgical processes.

FIG. 12 illustrates operating room smart system 54232, which may have an (e.g., dynamic) association with the surgical systems data set 54208. Included with the data set may be the smart surgical module 54262, a repository that may store, process, and manage surgical details and parameters. The module may enable integration with surgical tools, protocols, and specificities pertinent to legacy devices.

The camera 54276, may be linked to the operating room smart system 54232. The camera 54276 may be equipped with optical sensors and computational algorithms enabling the camera to scan the surgical room environment. The camera 54276 may interpret and discern characteristics of legacy devices 54206 present in its field of view.

The camera 54276 may recognize and analyze data visualizations on the legacy devices 54206. Through a combination of pattern recognition, optical character recognition (OCR), and/or machine learning models, etc., the camera may ascertain if a legacy device is actively presenting data relevant to the ongoing surgical procedure. The data may include vital metrics, graphical representations, or real-time feedback, etc.

When the relevant data is identified on the legacy device, the operating room smart system 54232 may conduct actions to access the relevant data. The smart system may capture, process, and/or project the data onto the smart system display 54210. The display, which may include high-resolution graphics (e.g., higher resolution than the legacy device) and customizable interface options, may present the data in a more digestible, interactive, and/or relevant format (e.g., than the legacy device), aiding surgical professionals in real-time decision-making.

The operating room smart system 54232 may employ multiple channels or protocols to enable data fidelity and communication. From data acquisition to visualization, error-checking, validation, and security measures may be in place to enable data integrity and relevance to the surgical context.

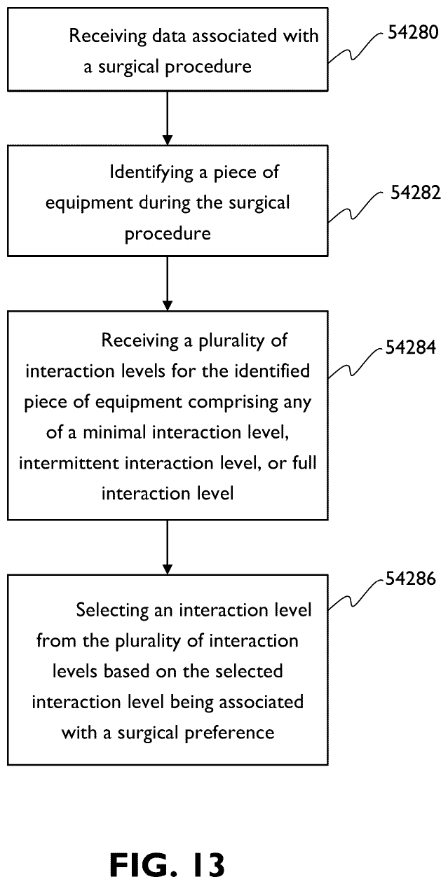

FIG. 13 illustrates an example block diagram of a surgical smart system. The surgical smart system, when implemented in an operating room, may modify the interaction between surgical staff and equipment. At 54280, the system may receive data associated with a surgical procedure.

At 54282, during the course of the procedure, the system may identify a piece of equipment in use. The identification may be based on direct input from the surgical team, sensor data, etc. When the equipment is identified, at 54284, the system may receive interaction levels for the piece of equipment. The interaction levels may be categorized as minimal, intermittent, or full interaction levels.

The surgical smart system's algorithms may process the interaction levels in the context of the provided surgical data. In examples, by cross-referencing the identified equipment with the surgical manifest, the system may determine the best use case scenario for the equipment in the ongoing procedure.

At 54286, the interaction levels associated with a piece of equipment may not be static. The system may provide flexibility, allowing for the selection of an interaction level from the interaction levels based on the selected interaction level being associated with a surgical preference. The dynamic adjustment may enable positive equipment functionality and surgical outcomes.

The system's interface may display the interaction levels for the surgical staff's reference. The distinct visualization of the interaction level may correlate with the best use case of the identified equipment. The visualization may aid in quick decision-making, allowing surgical staff to select the most appropriate interaction level from the display directly.

As the surgical procedure progresses, the surgical smart system may (e.g., continuously) monitor the operation of the identified equipment. The surgical smart system may gather operational data, which may include metrics like operational time, power consumption, performance metrics, etc.

The continuous monitoring may extend beyond data collection. The system may actively analyze the operational data to detect deviations or performance changes in the equipment. The continuous monitoring may enable (e.g., immediate) intervention, should an equipment malfunction or not operate as intended (or in a manner not beneficial for the patient).

Modules within the system may process the operational data to deduce the impact of the equipment's operation on patient physiological parameters. If a performance change is detected, the system may modify the interaction level (e.g., dynamically), enabling patient safety and positive procedure outcomes.

Predictive analytics may be used by the system. By scrutinizing the usage patterns of the identified equipment, the system may forecast the future performance of the equipment. The predictions may enable pre-emptive measures, minimizing surgical procedure interruptions.

For record-keeping and future reference, the system may display the analyzed operational data, which may include parameters like estimated time to failure for equipment. The data may not be confined to the system alone. The system may transmit the analyzed operational data to an external database (e.g., forming a comprehensive digital record of the equipment's operation during the surgical procedure).

In examples where a direct correlation between received data and predefined parameters is sought, the surgical smart system may employ a lookup table. The table may store pre-characterized information and, when queried with specific inputs, return the corresponding data. The mechanism may enable the retrieval of data sets or parameters (e.g., facilitating decision-making during procedures).

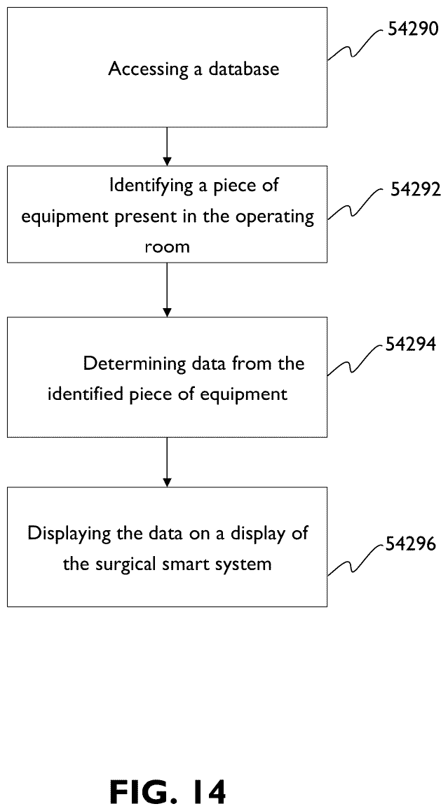

FIG. 14 illustrates a method for operating a surgical smart system in an operating room. At 54291, a database may be accessed. The database may include a surgical manifest and a lookup table of surgical equipment which may serve as a repository for storing and retrieving information regarding surgical assets and their respective specifications.

At 54292, a piece of equipment present in the operating room may be identified. Utilizing a camera of the surgical smart system, the identification of the piece of equipment may include comparing the piece of equipment with the information included in the database. The comparison may facilitate the recognition and verification of the equipment's presence and characteristics within the operating environment.

At 54294, data may be determined from the identified piece of equipment. The determination may be executed using Optical Character Recognition (OCR) (e.g., which may enable the extraction of text or numeral data from the images captured by the camera of the surgical smart system). The OCR may, for example, aid in recognizing identification numbers, labels, or data inscribed on the equipment.

At 54296, the data may be displayed on a display of the surgical smart system. The displayed data may include information extracted from the piece of equipment, which may provide the surgical personnel with details regarding the equipment's specifications, operational status, or other relevant data that may be associated with the surgical procedure.

A degree of interactivity between the surgical smart system and the piece of equipment may be quantified. The operation of the surgical smart system may be modified based on the quantified degree of interactivity. The quantification and modification may affect the utilization and coordination of the equipment associated with the surgical smart system.

The surgical manifest within the lookup table may be updated using the data from the identified piece of equipment, and the updated surgical manifest may be saved in the database. The operations may contribute to keeping the surgical manifest updated and accurate, reflecting the (e.g., most recent) data regarding the equipment present in the operating room.

The identified piece of equipment may be cross-referenced with the surgical manifest, and an alert may be generated on a condition that the piece of equipment is not found in the surgical manifest (e.g., serving as a safeguard, verifying that authorized or suitable equipment is utilized within the surgical procedure).

An operational status of the piece of equipment may be determined using the OCR-determined data, displaying the operational status on the display of the surgical smart system, and generating an alert on a condition that the operational status indicates that the identified piece of equipment is not suitable for use. The operations may affect the safety of the surgical procedures by indicating the readiness and appropriateness of the equipment in use.

The feasibility of wired communication with the piece of equipment may be determined, and instructions may be provided via a user interface of the surgical smart system for connecting the surgical smart system to the piece of equipment based on the determination that wired communication with the piece of equipment is possible. An operation of any of the surgical smart system or the piece of equipment may be adjusted based on the user connecting the surgical smart system to the piece of equipment.

1. A method for operating a surgical smart system in an operating room, the method comprising:
receiving data associated with a surgical procedure, wherein the data comprises at least a surgical manifest of the surgical procedure and a lookup table of surgical devices;
identifying a piece of equipment during the surgical procedure;
receiving a plurality of interaction levels for the identified piece of equipment comprising any of a minimal interaction level, intermittent interaction level, or full interaction level; and
selecting an interaction level from the plurality of interaction levels based on the selected interaction level being associated with a surgical preference.

2. The method of claim 1, wherein selecting the interaction level from the plurality of interaction levels based on the selected interaction level being associated with a surgical preference further comprises:
cross-referencing the identified piece of equipment with the surgical manifest;
determining a best use case of the identified piece of equipment based on the surgical manifest; and
selecting the interaction level from the plurality of interaction levels based on the best use case of the identified piece of equipment.

3. The method of claim 1, wherein selecting the interaction level from the plurality of interaction levels based on the selected interaction level being associated with a surgical preference further comprises:
cross-referencing the identified piece of equipment with the surgical manifest;
determining a best use case of the identified piece of equipment based on the surgical manifest;
displaying the plurality of interaction levels on a display of the surgical smart system, wherein the interaction level that is associated with the best use case is distinctly displayed among the plurality of interaction levels; and
receiving, from the display of the surgical smart system, the selected interaction level.

4. The method of claim 1, wherein the method further comprises:
monitoring operation of the identified piece of equipment during the surgical procedure;
generating operational data associated with the operation of the identified piece of equipment during the surgical procedure;
analyzing the operational data associated with the operation to detect a performance change in the identified piece of equipment;
displaying, on an interface of the surgical smart system, the analyzed operational data associated with the monitored operation; and
transmitting, to a database, the analyzed operational data associated with the monitored operation for further usage.

5. The method of claim 4, wherein analyzing the operational data associated with the monitored operation comprises determining an impact of the operation of the identified piece of equipment on patient physiological parameters, and wherein the method further comprises:
modifying the interaction level based on detecting the performance change in the identified piece of equipment.

6. The method of claim 4, wherein analyzing the data associated with the monitored operation comprises predicting a future performance of the identified piece of equipment based on detected usage patterns of the identified piece of equipment, and wherein the method further comprises:
modifying the interaction level based on the detected usage patterns of the identified piece of equipment.

7. The method of claim 4, wherein displaying the analyzed operational data associated with the monitored operation comprises displaying an estimated time to failure of the identified piece of equipment.

8. The method of claim 4, wherein transmitting the analyzed operational data associated with the monitored operation to a database comprises generating a comprehensive digital record of the operation of the identified piece of equipment during the surgical procedure.

9. A surgical smart system in an operating room, the surgical smart system comprising:
a processor configured to:
receive data associated with a surgical procedure, wherein the data comprises at least a surgical manifest of the surgical procedure and a lookup table of surgical devices;
identify a piece of equipment during the surgical procedure;
receive a plurality of interaction levels for the identified piece of equipment comprising any of a minimal interaction level, intermittent interaction level, or full interaction level;
select an interaction level from the plurality of interaction levels based on the selected interaction level being associated with a surgical preference.

10. The surgical smart system of claim 9, wherein the processor configured to select the interaction level from the plurality of interaction levels based on the selected interaction level being associated with a surgical preference comprises the processor being further configured to:
cross-reference the identified piece of equipment with the surgical manifest;
determine a best use case of the identified piece of equipment based on the surgical manifest; and
select the interaction level from the plurality of interaction levels based on the best use case of the identified piece of equipment.

11. The surgical smart system of claim 9, wherein the processor being configured to select the interaction level from the plurality of interaction levels based on the selected interaction level being associated with a surgical preference comprises the processor being further configured to:
cross-reference the identified piece of equipment with the surgical manifest;
determine a best use case of the identified piece of equipment based on the surgical manifest;
display the plurality of interaction levels on a display of the surgical smart system, wherein the interaction level that is associated with the best use case is distinctly displayed among the plurality of interaction levels; and
receive, from the display of the surgical smart system, the selected interaction level.

12. The surgical smart system of claim 9, wherein the processor is further configured to:
monitor operation of the identified piece of equipment during the surgical procedure;
generate operational data associated with the operation of the identified piece of equipment during the surgical procedure;
analyze the operational data associated with the operation to detect a performance change in the identified piece of equipment;
display, on an interface of the surgical smart system, the analyzed operational data associated with the monitored operation; and
transmit, to a database, the analyzed operational data associated with the monitored operation for further usage.

13. The surgical smart system of claim 12, wherein the processor configured to analyze the operational data associated with the monitored operation comprises the processor being configured to:
determine an impact of the operation of the identified piece of equipment on patient physiological parameters; and
modify the interaction level based on detecting the performance change in the identified piece of equipment.

14. The surgical smart system of claim 12, wherein the processor configured to analyze the operational data associated with the monitored operation comprises the processor being configured to:
predict a future performance of the identified piece of equipment based on detected usage patterns of the identified piece of equipment; and
modify the interaction level based on the detected usage patterns of the identified piece of equipment.

15. The surgical smart system of claim 12, wherein the processor configured to transmit the analyzed operational data associated with the monitored operation to a database comprises the processor being configured to generate a comprehensive digital record of the operation of the identified piece of equipment during the surgical procedure.

16. A surgical smart system in an operating room, the surgical smart system comprising:
a processor configured to:
receive data associated with a surgical procedure, wherein the data comprises at least a surgical manifest of the surgical procedure;
identify a piece of equipment;
receive a plurality of interaction levels for the identified piece of equipment comprising any of a minimal interaction level, intermittent interaction level, or full interaction level;
select an interaction level from the plurality of interaction levels.

17. The surgical smart system of claim 16, wherein the processor configured to select the interaction level from the plurality of interaction levels comprises the processor being further configured to:
cross-reference the identified piece of equipment with the surgical manifest;
determine a best use case of the identified piece of equipment based on the surgical manifest; and
select the interaction level from the plurality of interaction levels based on the best use case of the identified piece of equipment.

18. The surgical smart system of claim 16, wherein the processor being configured to select the interaction level from the plurality of interaction levels comprises the processor being further configured to:
cross-reference the identified piece of equipment with the surgical manifest;
determine a best use case of the identified piece of equipment based on the surgical manifest;
display the plurality of interaction levels on a display of the surgical smart system, wherein the interaction level that is associated with the best use case is distinctly displayed among the plurality of interaction levels; and
receive, from the display of the surgical smart system, the selected interaction level.

19. The surgical smart system of claim 16, wherein the processor is further configured to:
monitor operation of the identified piece of equipment during the surgical procedure;
generate operational data associated with the operation of the identified piece of equipment during the surgical procedure;
analyze the operational data associated with the operation to detect a performance change in the identified piece of equipment;
display, on an interface of the surgical smart system, the analyzed operational data associated with the monitored operation; and
transmit, to a database, the analyzed operational data associated with the monitored operation for further usage.

20. The surgical smart system of claim 19, wherein the processor configured to analyze the operational data associated with the monitored operation comprises the processor being configured to:
determine an impact of the operation of the identified piece of equipment on patient physiological parameters; and
modify the interaction level based on detecting the performance change in the identified piece of equipment.
